(19) United States
(12) Patent Application Publication
(10) Pub. No.: US 2010/0239650 A1
(43) Pub. Date: Sep. 23, 2010

(54) ISOTHIAZOLIN BIODELIVERY SYSTEMS

(76) Inventors: Wilson Kurt Whitekettle, Jamison, PA (US); Gloria Jean Tafel, Doylestown, PA (US)

Correspondence Address: General Electric Company;GE Global Patent Operation, 2 Corporate Drive, Suite 648, Shelton, CT, 06484, US

(21) Appl. No.: 12/407,953

(22) Filed: Mar. 20, 2009

(51) Int. Cl. A61K 9/127; A61K 31/425

(52) U.S. Cl. 424/450; 514/372

(57) ABSTRACT

A biodelivery system has been found which increases the efficiency and effectiveness of introducing antimicrobial compounds into complex biofilm matrices through the use of liposome carriers, thereby removing the biofouling in industrial water bearing systems, including piping, heat exchanges, condensers, filtration systems and fluid storage tanks.

According to one embodiment of the invention, antimicrobial compound containing liposomes are added to water systems prone to biofouling and biofilm formation. The liposomes, being similar in composition to microbial membranes or cells, are readily incorporated into the existing biofilm. Once the antimicrobial compound containing liposomes become entrained with the biofilm matrix, the decomposition or disintegration of the liposome proceeds. Thereafter the biocidal core is released to react directly with the biofilm encased microorganisms. Upon the death of the organisms, the matrix decomposes and thereby results in reduced fouling of the water bearing system, resulting in increased heat transfer, increased flux, less deposit of colloidal and particulate solids and dissolved organics on the surface of the microfiltration membrane, thereby reducing the frequency and duration of the membrane cleaning and ultimate replacement.

### FIELD OF THE INVENTION

The field of the invention generally relates to biodelivery systems for providing products or compounds, such as chemicals, to industrial systems. The invention also relates to compositions for use in a targeted delivery of said compositions to bacterial biofilms various environments.

### BACKGROUND OF THE INVENTION

Bacterial biofilms exist in natural, medical, and industrial environments. The biofilms offer a selective advantage to microorganisms to ensure the microorganisms' survival or to allow them a certain time to exist in a dormant state until suitable growth conditions arise. Unfortunately, this selective advantage poses serious threats to health, or to the efficiency and lifetime of industrial systems. The biofilms must be minimized or destroyed to improve the efficiency of industrial systems, or remove the potential health threats.

Many industrial or commercial operations rely on large quantities of water for various reasons, such as for cooling systems, or said systems may produce large quantities of wastewater, which result in the creation of biofilms that need to be treated. These industries include, but are not limited to, agriculture, petroleum, oil drilling, oil pipelines, oil storage, gas drilling, gas pipelines, gas storage, chemical, pharmaceutical, mining, metal plating, textile, papermaking, brewing, food and beverage processing, and semiconductor industries. In these operations, naturally occurring biofilms are continuously produced and often accumulate on numerous structural or equipment surfaces or on natural or biological surfaces. In industrial settings, the presence of these biofilms causes a decrease in the efficiency of industrial machinery, requires increased maintenance and presents potential health hazards. An example is the surfaces of water cooling towers which become increasingly coated with microbially produced biofilm slime which constricts water flow and reduces heat exchange capacity. Specifically, in flowing or stagnant water, biofilms can cause serious problems, including pipeline blockages, corrosion of equipment by growth of underfilm microbes and the growth of potentially harmful pathogenic bacteria. Water cooling tower biofilms may form a harbor or reservoir that perpetuates growth of pathogenic microorganisms such as Legionella pneumophila.

Another example of industrial systems are those systems that are found in the food and beverage industries. Food preparation lines are routinely plagued by biofilm build-up both on the machinery and on the food product where biofilms often include potential pathogens. Industrial biofilms, such as those found in the food industry, are complex assemblages of insoluble polysaccharide-rich biopolymers, which are produced and elaborated by surface dwelling microorganisms. More particularly, biofilms or microbial slimes are composed of polysaccharides, proteins and lipopolysaccharides extruded from certain microbes that allow them to adhere to solid surfaces in contact with water environments and form persistent colonies of sessile bacteria that thrive within a protective film. The film may allow anaerobic species to grow, producing acidic or corrosive conditions. To control these problems, processes and antimicrobial products are needed to control the formation and growth of biofilms. Control of biofilms involves the prevention of microbial attachment and/or the removal of existing biofilms from surfaces. While removal in many contexts is accomplished by short cleansing treatments with highly caustic or oxidizing agents, the most commonly used materials to control biofilms are biocides and dispersants. In U.S. Pat. No. 5,411,666, a method of removing a biofilm or preventing buildup of a biofilm on a solid substrate is taught, that comprises a combination of at least two biologically produced enzymes, such as an acidic or alkaline protease and a glucoamylase or alpha amylase and at least one surfactant. U.S. Pat. No. 6,759,040 teaches a method for preparing biofilm degrading, multiple specificity, hydrolytic enzyme mixtures that are targeted to remove specific biofilms.

U.S. Pat. No. 6,267,897, relates to a method of inhibiting biofilm formation in commercial and industrial water systems by adding one or more plant oils to the system. However, although the biocides are effective in controlling dispersed microorganism suspensions, i.e. planktonic microbes, biocides do not work well against sessile microbes, the basis of biofilms. This is due to the fact that biocides have difficulty penetrating the polysaccharide/protein slime layers surrounding the microbial cells. Thicker biofilms see little penetration of biocides and poor biocide efficacy is the result. One known method of trying to better control biofilms has been the addition of dispersants and wetting agents to biocide compositions to enhance biocide efficacy. Biodispersants may operate to keep planktonic microbes sufficiently dispersed so that they do not agglomerate or achieve the local densities necessary to initiate the extracellular processes responsible for anchoring to a surface, or initiating film- or colony-forming mechanisms. As components in biocidal treatment formulations, these biodispersants have helped in opening channels in the biofilm to allow better permeability of the toxic agents and to better disperse the microbial aggregates and clumps that have been weakened and released from the surfaces. However, biodispersants have proven to be more effective in preventing initial biofilm formation than in removing existing biofilms. In many cases, the activity of biodispersants has been responsible for only 25 to 30% biomass removal from biofouled surfaces, even when used in conjunction with a biocidal agent.

Therefore, a clear need still exists for an efficient and effective means for delivering antimicrobial compounds that are better able to penetrate existing biofilms and biofilm matrices, and more effective in killing microorganisms contained within a biofilm matrix, thus killing and eliminating biofilm, as well as preventing future formation nor buildup of biofilm, in systems, such as industrial systems. Decreasing the fouling of microfiltration systems, and providing less frequent cleaning and/or replacement which would enhance the overall filtration process, are also needs which should be addressed.

### SUMMARY OF THE INVENTION

A biodelivery system has been found which increases the efficiency and effectiveness of introducing antimicrobial compounds into complex biofilm matrices, through the use of liposome carriers, which can be used in natural, medical and industrial applications. In industrial applications, the delivery system can minimize or eliminate fouling in industrial systems, including, but not limited to, aqueous systems, such as piping, heat exchangers, condensers, filtration systems and media, and fluid storage tanks.

According to one embodiment of the invention, liposomes containing an antimicrobial agent, such as a hydrophilic biocide, are added to a water system prone to biofouling and biofilm formation. The liposomes, being similar in composition to the outer surface of the microbial cell wall structure or to the material on which the microbes feed, are readily incorporated into the microbes present in the existing biofilm. Once the liposomes become entrained with the biofilm matrix, digestion, decomposition or degradation of the liposome proceeds, releasing the antimicrobial agent, or biocidal aqueous core reacts locally with the biofilm—encased microorganisms. Upon the death of the organisms, the polysaccharide/protein matrix cannot be replenished and decomposes and thereby results in reduced bio fouling of the water bearing system. Depending on the particular system involved, this biofilm removal or destruction therefore results in increased heat transfer (industrial heat exchanger), increased flux (filter or filtration membrane), less deposit of colloidal and particulate solids and dissolved organics on the surface of the microfiltration membrane, thereby reducing the frequency and duration of the membrane cleaning and ultimate replacement, or general reduction of corrosive surface conditions in pipelines, tanks, vessels or other industrial equipment.

An alternate embodiment of the invention provides for a delivery system of actives into a natural, medical or industrial system, which can be chosen from the group consisting of anti-corrosion treatments, pesticides for agriculture and commercial home uses, food additives and preservatives, chemical and biological detection, color and flavor enhancement, odor control and aquatic pest management.

The various features of novelty that characterize the invention are pointed out with particularity in the claims annexed to and forming a part of this disclosure. For a better understanding of the invention, its operating advantages and benefits obtained by its uses, reference is made to the accompanying drawings and descriptive matter. The accompanying drawings are intended to show examples of the invention. The drawings are not intended as showing the limits of all of the ways the invention can be made and used. Changes to and substitutions of the various components of the invention can of course be made. The invention resides as well in sub-combinations and sub-systems of the elements described, and in methods of using them.

### BRIEF DESCRIPTION OF THE DRAWINGS

Refer now to the figures, which are meant to be exemplary and not limiting, and wherein like elements are numbered alike, and not all numbers are repeated in every figure for clarity of the illustration.

FIG. 1 is chart setting forth results obtained from an isothiazolin according to one embodiment of the invention.

FIG. 2 is chart setting forth further results obtained from an isothiazolin according to one embodiment of the invention.

FIG. 3 is chart setting forth results obtained from an isothiazolin according to one embodiment of the invention.

### DETAILED DESCRIPTION OF THE INVENTION

Approximating language, as used herein throughout the specification and claims, may be applied to modify any quantitative representation that could permissibly vary without resulting in a change in the basic function to which it is related. Accordingly, a value modified by a term or terms, such as “about”, is not limited to the precise value specified. In at least some instances, the approximating language may correspond to the precision of an instrument for measuring the value. Range limitations may be combined and/or interchanged, and such ranges are identified and include all the sub-ranges included herein unless context or language indicates otherwise. Other than in the operating examples or where otherwise indicated, all numbers or expressions referring to quantities of ingredients, reaction conditions and the like, used in the specification and the claims, are to be understood as modified in all instances by the term “about”.

As used herein, the terms “comprises,” “comprising,” “includes,” “including,” “has,” “having” or any other variation thereof, are intended to cover a non-exclusive inclusion. For example, a process, method, article or apparatus that comprises a list of elements is not necessarily limited to only those elements, but may include other elements not expressly listed or inherent to such process, method article or apparatus.

A delivery system has been found which increases the efficiency and effectiveness of introducing antimicrobial compounds into complex biofilm matrices through the use of liposome carriers, which can be used in natural, medical and industrial applications. In industrial applications, the delivery system can minimize or eliminate fouling in industrial systems, including, but not limited to, aqueous systems, such as cooling towers, piping, heat exchangers, condensers, filtration systems and media, and fluid storage tanks.

According to one embodiment of the invention, liposomes containing a biocidal or antimicrobial agent or compound are added to an industrial system prone to biofouling and biofilm formation. The liposomes, being similar in composition to microbial membranes or cells, are readily incorporated into the existing biofilm. Once the antimicrobial compound-containing liposomes diffuse into, adsorb or otherwise become entrained with the biofilm matrix, the microorganisms existing within the biofilm matrix will ingest the liposome structure, resulting in the decomposition or disintegration of the liposome inside the intracellular matrix of the microorganism, thereby releasing the antimicrobial compound into the intracellular matrix of the microorganism, ultimately resulting in the death of the microorganism. That is lipid decomposition and biocide release can be programmed to occur by making the lipid matrix sensitive to pH, redox potential, Ca⁺² concentration, or other changes. Thereafter the biocidal component that may be concentrated in the aqueous core of the liposome or in the lipid membrane portion of the liposome, is released to react directly with the biofilm-encased microorganisms. Thus, rather than adding a biocide at high levels to the bulk water system, a small quantity of liposome-encased biocide is taken up by the biofilm or by free (planktonic) organisms, and degradation of the liposome releases the biocide locally in or at the target organisms or their film matrix niche. The biocide thus attains a high concentration locally to kill the target organisms, and upon the death of the organisms, the polysaccharide/protein matrix that forms the biofilm cannot be maintained or regenerated and decomposes, and thereby results in reduced fouling of the water bearing system, resulting in increased heat transfer, increased flux, less deposit of colloidal and particulate solids and dissolved organics on the surface of the microfiltration membrane, thereby reducing the frequency and duration of the membrane cleaning and ultimate replacement or other benefits.

Liposomes, or lipid bodies, are systems in which lipids are added to an aqueous buffer to form vesicles, structures that enclose a volume. The liposomes may be comprised of lipids selected from the group consisting of phospholipids, lethicin, phosphatidyl choline, glycolipid, triglyceride, sterol, fatty acid, sphingolipid, or combinations thereof.

More specifically, liposomes are microscopic vesicles, most commonly composed of phospholipids and water. The liposomes may be made from phospholipids derived from various sources, including, but not limited to soybeans and eggs. When properly mixed, the phospholipids arrange themselves into a bilayer or multilayers, very similar to a cell membrane, surrounding an aqueous volume core. Liposomes can be produced to carry various compounds or chemicals within the aqueous core, or the desired compounds can be formulated in a suitable carrier to enter the lipid layer(s). Liposomes can be produced in various sizes and may be manufactured in submicron to multiple micron diameters. The liposomes may be manufactured by several known processes. Such processes include, but are not limited to, controlled evaporation, extrusion, injection, microfluid processors and rotor-stator mixers. Liposomes can be produced in diameters ranging from about 10 nanometers to greater than about 15 micrometers. When produced in sizes from about 100 nanometers to about 2 micrometer sizes the liposomes are very similar in size and composition to most microbial cells. The biocide or antimicrobial compound containing-liposomes should be produced in sizes that mimic bacterial cells, for example, from about 0.05 to about 15μ, or alternately, about 0.1 to 10.0μ.

In one embodiment, effective amounts of the biocide containing liposome is introduced into an industrial system which is prone to biofouling and biofilm formation, or can be introduced into systems that already exhibit signs of biofouling or biofilm formation. The effective amount will vary according to the antimicrobial compound or biocide, and the aqueous system to which it is added, but one embodiment provides from about 0.01 ppm to about 100 ppm, with an alternative of from about 0.05 to about 50 ppm, alternately from about 0.05 to about 5.0 The liposomes, being similar in composition to microbial membranes, or cell walls, are readily incorporated into the existing biofilm and become entrained within the biofilm matrix. The liposomes containing biocides have improved penetration of the biofilm matrix, due to similarity in composition and structure with the biofilm. Once the liposome is incorporated or entrained within the existing biofilm matrix, the liposome will begin to disintegrate. Upon the decomposition or programmed disintegration of the liposome, the biocidal compound contained within the aqueous core of the liposome is released to react directly with the biofilm encased microorganisms, resulting in their demise. Upon the death of the organisms, the polysaccharide/protein matrix will rapidly decompose, freeing the surface from contaminating microbes.

A principal feature of one embodiment of the present invention is that the liposomes constitute extremely small hydrophobic bodies that may readily survive in and disperse in systems, such as for example, aqueous or natural systems, and yet will adsorb to or penetrate a biofilm and preferentially target or be targeted by the microbes that inhabit, constitute or sustain the biofilm. As such, the liposomes deliver a biocidal agent directly to the microbes or biofilm, resulting in effective locally biocidal level of activity, without requiring that the industrial system as a whole sustain a high dose. Thus, where conventional biofilm treatment may require dosing with a bulk biocidal chemical at a certain level, delivery via liposome may be dosed at levels an order of magnitude or more lower in the aqueous system, yet still achieve, or build up to a level that effectively controls or removes biofilm. This lower level of biocide concentration has positive effects on the environment due to the efficacy resulting from the delivery system. Additionally, depending upon the particular system that is being treated, an embodiment provides for flexibility in where the liposomes are actually delivered into the system. If there is one particular area in a system that is prone to biofilm creation, the delivery of the liposomes may be delivered to that particular portion or point of the system, such that the delivery of the biodelivery composition is to a targeted location, and not necessarily privy to or exposed to the entire system. As smaller doses of the liposome containing biocides are needed due to the efficacy of the biocides in this format, an entire system or process need not be flooded with or treated with biocides.

Indeed, while the terms “antimicrobial” or “biocide” or “biocidal” have been employed to describe the agent carried by the liposome, these agents need not be the highly bioactive materials normally understood by those terms, but may include a number of relatively harmless materials that become highly effective simply by virtue of their highly localized release. Thus, for example, surfactants or harmless ammonium or phosphonium halide salts, when released locally, may affect the normal action of extracellular colony-forming secretions, and are to be included as antimicrobial or biocidal agents for purposes of the invention, and the same mechanism may be employed to deliver other treatment chemicals to the targeted biofilm sites.

Aqueous systems that can be treated by this method include, but are not limited to, potable and non-potable water distribution systems, cooling towers, boiler systems, showers, aquaria, sprinklers, spas, cleaning baths, air washers, pasteurizers, air conditioners, fluid transporting pipelines, storage tanks, ion exchange resins, food and beverage processing lines, metalworking fluid baths, coal and mineral slurries, metal leaching fluids, wastewater treatment facilities, mollusk control, pulp and papermaking operations, acid mine drainage, or any application prone to biofouling by microbial species. Application such as oil drilling, oil storage tanks or oil pipelines, where biofilms form in stagnant or pooled aqueous sumps or lenses along the conduit system, may also be effectively treated.

Additional applications for liposome delivery of a treatment chemical comprise natural, medical and industrial systems, such as, but not limited to anti-corrosion treatments for equipment generally, delivery of hormone, vitamin or antioxidant treatments or antibiotic and gene therapies for medical or veterinary purposes, delivery of pesticides for agriculture and commercial home uses, effective formulations of food additives and preservatives, targeted delivery for chemical and biological detection systems, color and flavor enhancement, odor control, fungicides, rodenticides, insecticides, mildew control and aquatic pest management.

Various biocides, for example non-oxidizing biocides, can be incorporated into the liposome, which would be effective. The use of certain biocides has shown the efficacy of this delivery system versus inclusion of biocides in the industrial systems wherein the biocide is outside of the liposome delivery system. The level or concentration of biocides is measured in active levels, to provide consistency across various forms of the same biocide.

One embodiment of the invention calls for the use of isothiazolin-3-one biocides. These isothiazolin-3-one liposome formulations are more effective at killing and removing biofilms when compared to the same isothiazolin-3-one compounds at the same active concentrations, which are introduced into systems, but not incorporated in liposomes, as the liposome containing biocides readily penetrate the microbial biofilms and are highly effective at destroying the biofilm matrix. This liposome delivery method may comprise 5-chloro-2-methyl-4-isothizolin-3-one and 2-methyl-4-isothiazolin-3-one, but any substituted isothiazolin-3-one based biocide can be made significantly more effective when delivered in a liposome biodelivery system or composition.

An example of an isothiazolin-3-one compound is

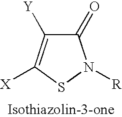

Where:

R═H, Cl, Br, I, CₙH₍ₙ₊₂₎

X═H, Cl, Br, I, CₙH₍ₙ₊₂₎

Y═H, Cl, Br, I, CₙH₍ₙ₊₂₎

For an embodiment of liposomes comprising isothiazolin, the active range is from about 0.02 to about 10.0 actives, and alternately from about 0.03 to about 5.5 active.

Liposomes of the present invention may be created as multi-layer bodies, in which one or more additional layers are provided to enhance the stability of the liposomes or to effectuate a programmed release of the underlying lipid body and contents. Thus, this technology may be used to encapsulate medicines for intracorporal delivery, such that the additional layers may include a protective layer that is hydrolyzed or otherwise breaks down over time to provide a sustained release or longer lifetime of the underlying liposome. Such additional layer may additionally or alternatively include an encapsulating polymer that selectively breaks down when the multi-layer liposome encounters a low-pH environment, like the corrosive high acidity environment that may develop beneath a biofilm. A layer may also be compounded to be vulnerable to sulfur-fixing bacteria, causing the liposome to specifically release its biocide in proximity to these corrosive organisms often present in a waste or pipeline system. Furthermore, several such layers may be employed to assure a sufficient lifetime of the liposome, preferably on the order of several days as well as an ability to target a specific niche or environment in the biofilm. This assures that the liposomes will effectively encounter the target organisms or biofilm colonies and deliver their biocides thereto. The lipid material itself may be treated to provide enhanced resistance to hydrolysis or decay, or the added layers may be formed of various hardenable or cross-linkable oils or polymers.

An alternate embodiment of the invention provides for a biodelivery composition for delivering at least one antimicrobial composition into a biofilm present in an industrial system, wherein the biofilm comprises at least one microorganism species; b) the biodelivery composition comprises a liposome structure containing at least one lipid or phospholipid type component; and c) the liposome structure encapsulates at least one antimicrobial composition.

A further embodiment provides for the targeted delivery of biocide actives into an industrial system, such as an industrial aqueous system, by introducing into said system an effective amount of said biocides in a critical area of said system. By targeting an area, and entry at a specific point in a process, the efficacy of the liposome system provides for a noteworthy impact on the environment as well as the cost of maintaining a system, as the entire system does not need to be flooded with biocides, only the specific area of interest.

The invention will now be described with respect to certain examples that are merely representative of the invention and should not be construed as limiting thereof.

### EXAMPLES

The invention is illustrated in the following non-limiting examples, which are provided for the purpose of representation, and are not to be construed as limiting the scope of the invention. All parts and percentages in the examples are by weight unless indicated otherwise.

### Example 1

Three batches of liposomes (150 nanometers average diameter) were created that incorporated an isothiazolin biocide, Kathon™ (available from Rohm & Haas, Philadelphia, Pa.) as the active ingredient. The liposomes were then placed in microtiter plates that had microbial biofilms coating them. The microbe inhibiting efficacy of the isothiazolin liposomes was then compared with non-liposomal isothiazolin biocide when used at the same isothiazolin concentrations. The liposomes containing isothiazolin penetrated the biofilm and inhibited the biofilm organisms much more effectively than the non-liposomal isothiazolin solution.

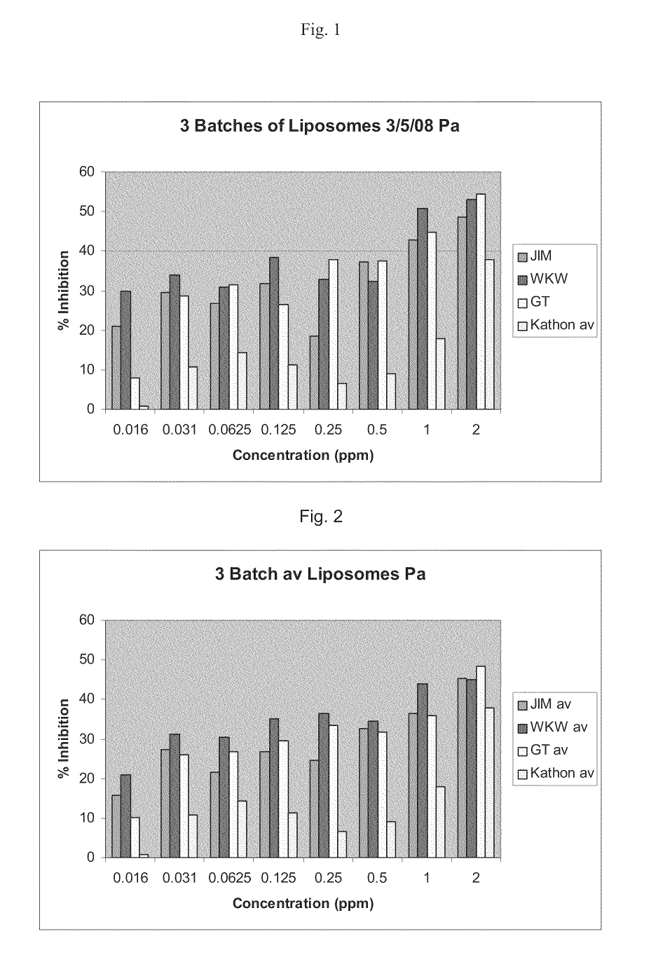

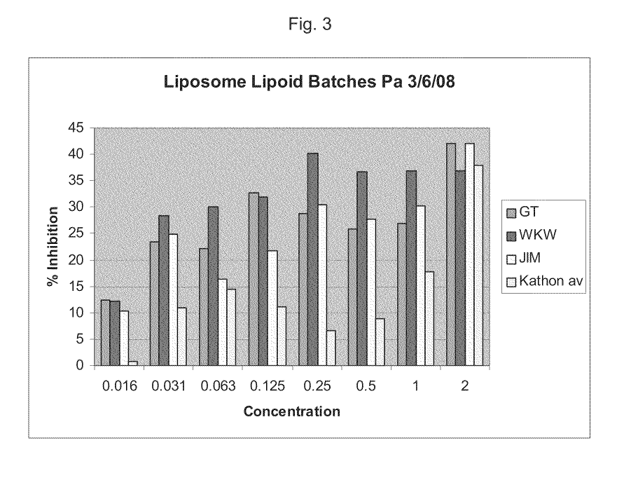

The results are shown in the tables below and in FIGS. 1, 2 and 3. The non-liposomal isothiazolin is listed as Kathon av, each of the liposome samples were made by three different technicians and are referred to by code. The tables and charts show the concentration of the isothiazolin versus the percent inhibition of the biofilm. It is clear from both the tables and the figures that in all three trials, the liposomal isothiazolin formulations exhibited more effective biofilm killing/removal efficiency than the isothiazolin control (listed as Kathon av) in every liposome concentration that was tested. The liposome carrier is highly effective at delivering biocide to the biofilm at low isothiazolin concentrations, thus providing better biofilm control at much reduced isothiazolin concentrations (reduced toxicity and cost performance).

TABLE 1

| Concentration | % inhibition | % inhibition | % inhibition | % inhibition |
|---|---|---|---|---|
| (ppm) | JIM | WKW | GT | Kathon av |
| 0016 | 21 | 29.9 | 7.9 | 0.93 |
| 0.031 | 29.7 | 34.1 | 28.7 | 10.9 |
| 0.0625 | 26.8 | 31 | 31.4 | 14.5 |
| 0.125 | 31.7 | 38.3 | 26.5 | 11.3 |
| 0.25 | 18.6 | 32.9 | 37.9 | 6.7 |
| 0.5 | 37.4 | 32.4 | 37.5 | 9.0 |
| 1 | 42.9 | 50.8 | 44.8 | 17.9 |
| 2 | 48.6 | 53.1 | 54.4 | 37.9 |

TABLE 2

| Concentration | % inhibition | % inhibition | % inhibition | % inhibition |
|---|---|---|---|---|
| (ppm) | JIM | WKW | GT | Kathon av |
| 0016 | 15.7 | 21.1 | 10.2 | 0.93 |
| 0.031 | 27.3 | 31.3 | 26.1 | 10.9 |
| 0.0625 | 21.6 | 30.5 | 26.8 | 14.5 |
| 0.125 | 26.7 | 35.1 | 29.6 | 11.3 |
| 0.25 | 24.6 | 36.6 | 33.4 | 6.7 |
| 0.5 | 32.6 | 34.6 | 31.8 | 9.0 |
| 1 | 36.6 | 43.9 | 35.9 | 17.9 |
| 2 | 45.3 | 45.1 | 48.3 | 37.9 |

TABLE 3

| Concentration | % inhibition | % inhibition | % inhibition | % inhibition |
|---|---|---|---|---|
| (ppm) | JIM | WKW | GT | Kathon av |
| 0016 | 10.4 | 12.3 | 12.4 | 0.93 |
| 0.031 | 24.9 | 28.4 | 23.5 | 10.9 |
| 0.0625 | 16.3 | 30 | 22.1 | 14.5 |
| 0.125 | 21.7 | 31.9 | 32.7 | 11.3 |
| 0.25 | 30.5 | 40.2 | 28.8 | 6.7 |
| 0.5 | 27.7 | 36.7 | 26.0 | 9.0 |
| 1 | 30.3 | 37.0 | 26.9 | 17.9 |
| 2 | 42.0 | 37.0 | 42.1 | 37.9 |

While the present invention has been described with references to preferred embodiments, various changes or substitutions may be made on these embodiments by those ordinarily skilled in the art pertinent to the present invention with out departing from the technical scope of the present invention. Therefore, the technical scope of the present invention encompasses not only those embodiments described above, but also all that fall within the scope of the appended claims.

What is claimed is:

1. A biodelivery composition for delivering at least one antimicrobial composition into a biofilm present in an industrial system, wherein
a) the biofilm comprises at least one microorganism species;
b) the biodelivery composition comprises a liposome structure containing at least one lipid or phospholipid type component; and
c) the liposome structure encapsulates at least one antimicrobial composition.

2. The biodelivery composition of claim 1 wherein the lipid is one member selected from the group consisting of phospholipids, lethicin, phosphatidyl choline, glycolipid, triglyceride, sterol, fatty acid, sphingolipid, or combinations thereof.

3. The biodelivery composition of claim 2 wherein the lipid is a phospholipid.

4. The biodelivery composition of claim 3 wherein the phospholipid is derived from soybeans or eggs.

5. The biodelivery composition of claim 2 wherein the lethicin is a mixture of lipids.

6. The biodelivery composition of claim 1 wherein the antimicrobial composition comprises at least one biocide.

7. The biodelivery composition of claim 6 wherein the antimicrobial composition comprises a non-oxidizing biocide.

8. The biodelivery composition of claim 6 wherein the biocide is an isothiazolin biocide.

9. The biodelivery composition of claim 8 wherein the isothiazolin biocide comprises at least one member chosen from the group consisting of 5-chloro-2-methyl-4-isothizolin-3-one, 2-methyl-4-isothiazolin-3-one, or any combinations thereof.

10. The biodelivery composition of claim 1 wherein the liposome structure is up to about 200 microns in diameter.

11. The biodelivery composition of claim 1 wherein the liposome structure is between about 500 nanometers to about 10 microns in diameter.

12. The biodelivery composition of claim 1 wherein the industrial system is an aqueous system.

13. The biodelivery composition of claim 12 wherein the industrial system is chosen from the group consisting of water distribution systems, cooling towers, boiler systems, showers, aquaria, sprinklers, spas, cleaning bath systems, air washers, pasteurizers, air conditioners, fluid transporting pipelines, storage tanks, ion exchange resins, food and beverage processing lines, paint spray booths, metalworking fluid baths, coal and mineral slurries, metal leaching fluids, wastewater treatment facilities, pulping and papermaking suspensions, mollusk control, acid mine drainage, oil drilling pipes, oil pipelines, oil storage tanks, gas drilling pipes, gas pipelines, or any industrial application prone to microbial induced biofilm formation or microbial induced corrosion.

14. A method for delivering an antimicrobial composition into a biofilm in an industrial system comprising the steps of:
a) forming a liposome structure which encapsulates at least one antimicrobial composition; and
b) introducing an effective amount of the liposomes of a) above to an industrial system that is prone to biofouling or biofilm formation.

15. The method of claim 14 wherein the liposome structures are introduced at from about 0.01 ppm to about 100 ppm.

16. The method of claim 14 wherein the liposome structures are introduced in the industrial system at a targeted location.

17. The method of claim 14 wherein the liposome structure comprises a biocide.

18. The method of claim 17 wherein the biocide is an isothiazolin biocide.

19. The method of claim 18 wherein the biocide comprises at least one member chosen from the group consisting of 5-chloro-2-methyl-4-isothizolin-3-one, 2-methyl-4-isothiazolin-3-one, or any combinations thereof.
